(19) United States
(12) Patent Application Publication
(10) Pub. No.: US 2004/0193050 A1
(43) Pub. Date: Sep. 30, 2004

(54) Ultrasonic transmitting and receiving apparatus

(75) Inventor: Eiji Ogawa, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 10/805,205

(22) Filed: Mar. 22, 2004

(30) Foreign Application Priority Data
Mar. 24, 2003 (JP) 2003-081317

(51) Int. Cl. A61B 8/00

(52) U.S. Cl. 600/438; 600/440; 600/443

(57) ABSTRACT

An ultrasonic transmitting and receiving apparatus capable of obtaining ultrasonic images with high quality and reduced effect of side lobes. The apparatus includes: an ultrasonic transducer array including plural elements for transmitting and receiving ultrasonic waves; a drive signal generating unit for generating drive signals for respectively driving the plural elements; a transmission control unit for controlling the drive signal generating unit such that transmitted ultrasonic waves form an ultrasonic beam transmitted in at least one direction; a reception control unit for performing reception focusing processing on plural detection signals obtained based on ultrasonic waves received by the plural elements so as to form a reception focal point in at least one direction thereby forming an ultrasonic beam; and a scanning control unit for changing directivity of plural ultrasonic components constituting the ultrasonic beam according to a sound ray direction of the ultrasonic beam.

### BACKGROUND OF THE INVENTION

1. Field of the invention

The present invention relates to an ultrasonic transmitting and receiving apparatus to be used for obtaining ultrasonic images by transmitting ultrasonic waves and receiving ultrasonic echoes.

2. Description of a Related Art

FIG. 13A shows the structure of an ultrasonic transducer array 100 that is generally used in a conventional ultrasonic transmitting and receiving apparatus. The ultrasonic transducer array 100 includes, for example, plural ultrasonic transducers 101 arranged one-dimensionally. The ultrasonic transducer 101 is an element for transmitting or receiving ultrasonic waves, and a piezoelectric element such as piezoelectric ceramic represented by PZT (Pb (lead) zirconate titanate) or a macromolecule piezoelectric element represented by PVDF (polyvinylidene difluoride) is used. Electrodes are formed on both ends of these ultrasonic transducers 101, and these electrodes are connected to drive signal generating circuits including pulsers etc., respectively. Applying a voltage to the ultrasonic transducer 101, the piezoelectric element expands and contracts by piezoelectric effect to generate ultrasonic waves. Accordingly, as shown in FIG. 13B, by driving plural ultrasonic transducers 101 at predetermined time intervals, spherical waves transmitted from the respective ultrasonic transducers 101 are synthesized and an ultrasonic beam having a focal point formed in a desired direction and a desired depth can be transmitted.

Now, in ultrasonic imaging, side lobes produced when transmitting ultrasonic beams become problematic. When an ultrasonic beam having directivity is transmitted, in spatial distribution of acoustic pressure intensity, a local maximum acoustic pressure region that occurs in the vicinity of the center axis in a transmitting direction is referred to as “main lobe”, and a local maximum acoustic pressure region that occurs in a direction other than that is referred to as “side lobe”. There are various factors of occurrence of the side lobes, and, for example, it is produced depending on the relationship between the element pitch of the ultrasonic transducers and ultrasonic frequency (such side lobe is referred to as “grating lobe”), or produced by the unwanted vibration of the ultrasonic transducers. Normally, an ultrasonic echo received by the ultrasonic transducer is subjected to signal processing as an echo that has propagated from the direction of the main lobe. On this account, in the case where the side lobe component is large or there is a strong reflector in the side lobe direction, an artifact (virtual image) is generated, and the image quality of the ultrasonic image is degraded.

Accordingly, in order to suppress such unwanted component, various measures have been taken. For example, improvement in the delay accuracy of the transmission beams, miniaturization of the element, and enlarging the aperture diameter have been performed so as to acuminate the form of the main lobe. Alternatively, transmission and reception of ultrasonic waves are performed while weighting the intensity distribution of elements constituting the array with Gaussian distribution (Gaussian apodization) or weighting the waveform on the time base. However, there are limits to these techniques, and the side lobes have not yet been reduced to the sufficient level. Further, in the case where the ultrasonic beam is transmitted in a largely tilted condition, the level of the side lobe component becomes higher, and accordingly, it becomes more difficult to reduce it. Therefore, the effect on the image quality becomes a major problem.

U.S. Pat, No. 6,179,780 discloses the following technology for reducing the effect by the side lobes when plural ultrasonic beams are transmitted and received simultaneously. That is, a method of forming plural reception beams for one transmission beam, or a method of identifying transmission beams by changing frequencies of plural transmission beams or coding transmission beams by using Barker code, Golay code, etc. to relate them with received ultrasonic echoes are cited. Further, since there is a region called as “null line” where the acoustic pressure becomes generally zero between a main lobe and a side lobe, a method of performing alignment of the main lobe of another ultrasonic beam in the region, a method of simply separating the intervals of transmission beams, and a method of shifting the center frequencies of the transmission beams are also cited.

### SUMMARY OF THE INVENTION

The present invention has been achieved in view of the above-described problems. An object of the present invention is to reduce the side lobe component as an unwanted component other than the main lobe in an ultrasonic transmitting and receiving apparatus for acquiring ultrasonic images by transmitting and receiving ultrasonic waves.

In order to solve the above-described problems, an ultrasonic transmitting and receiving apparatus according to one aspect of the present invention comprises: an ultrasonic transducer array including plural ultrasonic transducers for transmitting ultrasonic waves and receiving ultrasonic waves reflected from an object to be inspected; drive signal generating means for generating drive signals for respectively driving the plural ultrasonic transducers; transmission control means for controlling the drive signal generating means such that ultrasonic waves to be transmitted from the plural ultrasonic transducers form a transmission beam to be transmitted in at least one direction; reception control means for performing reception focusing processing on plural detection signals obtained based on ultrasonic waves received by the plural ultrasonic transducers so as to form a reception focal point in at least one direction thereby forming a reception beam; and control means for changing directivity of plural ultrasonic components constituting the transmission beam in accordance with a sound ray direction in which the transmission beam is transmitted and/or changing directivity of plural ultrasonic components constituting the reception beam in accordance with a sound ray direction in which the reception focal point of the receiving beam is formed.

According to the present invention, since the directivity of the ultrasonic components constituting ultrasonic beams is controlled in accordance with the sound ray direction, ultrasonic beams with reduced side lobe component in any sound ray direction can be transmitted and received.

### BRIEF DESCRIPTION OF THE DRAWINGS

FIGS. 1A and 1B show acoustic pressure intensity profiles in the case where ultrasonic beams at small sound ray angles are formed by using unit beams having strong directivity and unit beams having weak directivity, respectively;

FIGS. 2A and 2B are diagrams for explanation of the principle of forming ultrasonic beams at small sound ray angles;

FIGS. 3A and 3B show acoustic pressure intensity profiles in the case where ultrasonic beams at large sound ray angles are formed by using unit beams having strong directivity and unit beams having weak directivity, respectively;

FIGS. 4A and 4B are diagrams for explanation of the principle of forming ultrasonic beams at large sound ray angles;

FIG. 5 is a block diagram showing the constitution of an ultrasonic transmitting and receiving apparatus according to the first embodiment of the present invention;

FIGS. 6A is a diagram for explanation of a normal drive method of elements, and FIG. 6B is a diagram for explanation of an effective aperture control method of elements;

FIG. 7 is a flowchart showing an ultrasonic transmitting and receiving method according to the first embodiment of the present invention;

FIG. 8A shows an acoustic pressure intensity profile of an ultrasonic beam at small sound ray angles obtained by using unit beams having strong directivity, and FIG. 8B shows an acoustic pressure intensity profile of an ultrasonic beam at small sound ray angles obtained by using unit beams having weak directivity;

FIG. 9A shows an acoustic pressure intensity profile of an ultrasonic beam at large sound ray angles obtained by using unit beams having strong directivity, and FIG. 8B shows an acoustic pressure intensity profile of an ultrasonic beam at large sound ray angles obtained by using unit beams having weak directivity;

FIG. 10 is a diagram for explanation of the aperture control method of elements;

FIGS. 11A to 11C are diagrams for explanation of an transmitting method of ultrasonic beams in an ultrasonic transmitting and receiving apparatus according to the second embodiment of the present invention;

FIG. 12A shows the state in which plural reception focal points are formed with respect to one transmitting direction of an ultrasonic beam, and FIG. 12B shows the state in which plural reception focal points are formed with respect to each of plural transmitting directions; and

FIG. 13A is a diagram showing the structure of a conventional ultrasonic transducer array and an ultrasonic beam transmitted therefrom, and FIG. 13B shows timing pulses to be applied to the respective ultrasonic transducers.

### DESCRIPTION OF THE PREFERRED EMBODIMENTS

Hereinafter, embodiments of the present invention will be described in detail by referring to the drawings. The same component elements are assigned with the same reference numerals and the descriptions thereof will be omitted. In the present application, from among components of transmitted or received ultrasonic beams, the component other than the component (main lobe), that is transmitted or received in an intended direction, is referred to “side lobe component”, which includes an acoustic floor, side lobe, grating lobe, etc. Further, the propagation direction of a transmission beam, which is formed by ultrasonic waves transmitted in adjusted phases from plural ultrasonic transducers and propagates in a desired direction, is referred to as a “sound ray direction”. Also, the propagation direction of a reception beam, which is obtained by synthesizing the phase matched detection signal of ultrasonic echoes received in plural ultrasonic transducers and propagates from a desired direction, is referred to as a “sound ray direction”.

FIGS. 1A to 4B are diagrams for explanation of the principle of transmitting and receiving ultrasonic beams in an ultrasonic transmitting and receiving apparatus according to the first embodiment of the present invention. In this embodiment, control of directivity is performed with respect to each of the plural ultrasonic waves that form transmission beams and reception beams. Generally, the “directivity” means sensitivity distribution when transmitting or receiving ultrasonic waves, and the condition with higher sensitivity to an arbitrary direction is represented as “directivity is strong” and the condition with sensitivity in broader directions is represented as “directivity is weak”.

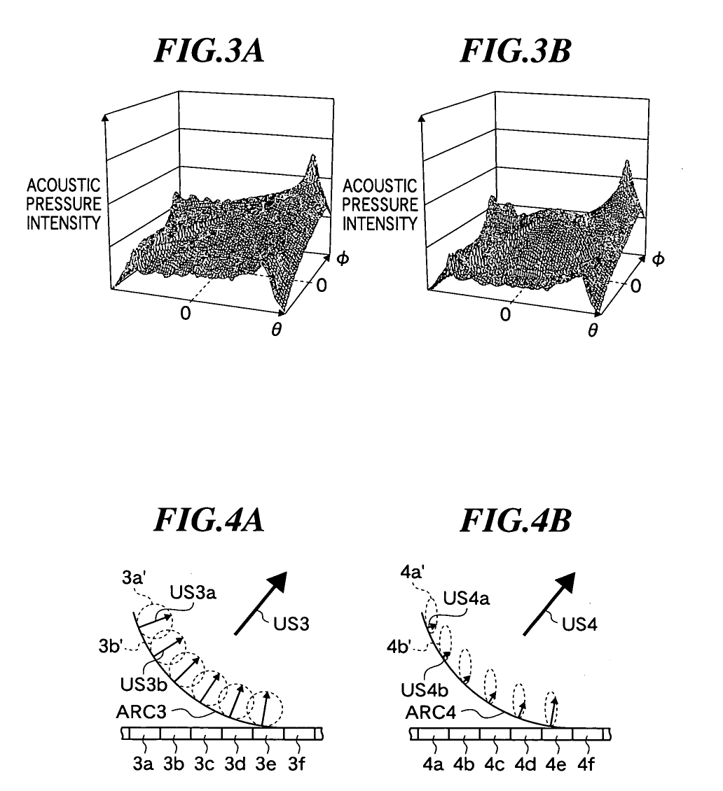

First, the relationship between the directivity of plural ultrasonic waves that form ultrasonic beams transmitted or received in a predetermined direction and the side lobe will be described by referring to FIGS. 1A to 4B. FIGS. 1A, 1B, 3A, and 3B show acoustic pressure intensity distribution (hereinafter, also referred to as “acoustic pressure intensity profile”) formed on an arbitrary focal plane within space by transmitting and receiving ultrasonic waves. These profiles are obtained by the simulation in which constituent factors of a two-dimensional ultrasonic transducer array, i.e., a number of the plural ultrasonic transducers, arrangement, pitch, aperture condition including an aperture diameter etc. and the waveform parameter are fixed and set to form a focal point in an arbitrary depth. In FIGS. 1A, 1B, 3A, and 3B, θ and φ represent angles of sound rays, the angle θ is an angle relative to a first surface orthogonal to the transmission and reception surface of the ultrasonic transducer array, and the angle φ is an angle relative to a second surface orthogonal to the transmission and reception surface and the first surface.

FIGS. 1A and 1B show acoustic pressure intensity profiles in the case where ultrasonic beams are formed by using the two-dimensional array toward the central axis direction of the array, that is, toward the direction at small sound ray angles. FIG. 1A shows the case of using plural ultrasonic transducers generating ultrasonic waves having weak directivity (element having weak directivity), and FIG. 1B shows the case of using plural ultrasonic transducers generating ultrasonic waves having strong directivity (element having strong directivity). As shown in FIGS. 1A and 1B, in the case where ultrasonic beams are formed in the direction at the small sound ray angle, the acoustic pressure intensity profile in which the side lobe component is entirely suppressed lower can be obtained by using the element having strong directivity (FIG. 1B).

The reason why the difference thus appears between the acoustic pressure intensity profiles depending on the directivity of the ultrasonic transducers is as follows.

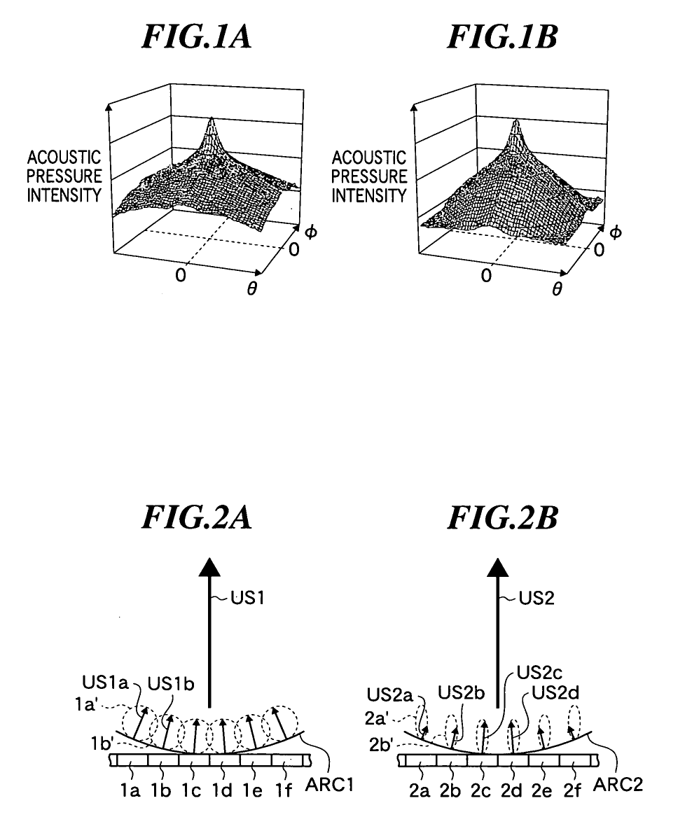

FIGS. 2A and 2B are diagrams for explanation of the principle of forming ultrasonic beams by using plural elements. An arc ARC1 and an arc ARC2 schematically show the drive timing with which plural elements 1a, 1b, . . . , and plural elements 2a, 2b, . . . are driven, respectively. As shown in FIGS. 2A and 2B, in the case where ultrasonic beams US1 and US2 are formed in the directions at small sound ray angles, plural elements 1a, 1b, . . . , and plural elements 2a, 2b, . . . are driven sequentially from both ends toward the center. Thereby, plural ultrasonic waves are generated, and their wavefront are synthesized to form ultrasonic beams.

In FIGS. 2A and 2B, broken lines are shown so that acoustic pressure intensity of the ultrasonic waves generated from each element is represented by the length of the chord drawn from the contact point of the respective broken line and the arc ARC1 or ARC2.

As shown in FIG. 2A, the ultrasonic waves 1a′, 1b′, generated from the elements 1a, 1b, . . . having weak directivity can be roughly regarded as spherical waves. In the ultrasonic waves 1a′, 1b′, . . . , ultrasonic components are isotropically diffused from the respective ultrasonic waves. On this account, the components US1a, US1b, . . . having the nearly uniform magnitudes from all of the elements contribute to formation of the ultrasonic beam US1 regardless of the arrangement of the elements 1a, 1b . . . Here, vectors US1a, US1b, . . . represent components that contribute to the ultrasonic beam US1 from among the generated ultrasonic beams 1a′, 1b′, . . . However, the components irrelevant to the contribution to the ultrasonic beam US1, that is, unwanted components are also diffused isotropically.

On the other hand, as shown in FIG. 2B, the ultrasonic waves 2a′, 2b′, . . . generated from the elements 2a, 2b, . . . having strong directivity include a large amount of components in a direction perpendicular to the ultrasonic transducer array surface. Here, the components that contribute to the formation of the ultrasonic beam US2 are US2a, US2b. On this account, in ultrasonic waves 2c′, 2d′ generated from the elements 2c, 2d disposed near the center of the ultrasonic transducer array, as shown by the components US2c and US2d, most components thereof contribute to the formation of the ultrasonic beam US2. Further, as the position of the element is farther from the vicinity of the center, as shown by the components US2a and US2e, the magnitude of components that contribute to the formation of the ultrasonic beam US2 becomes smaller gradually. However, in the case where the sound ray angles of the ultrasonic beam US2 are small, since angles formed by the components US2a and US2f that contribute to the formation of the ultrasonic beam US2 and transmitting directions (perpendicular line directions of the respective elements) of the ultrasonic waves 2a′ and 2f′ become not so much larger in both ends of the ultrasonic transducer array (for example, elements 2a and 2f), the magnitude of the components US2a and US2e are never reduced largely. On the other hand, in the case where the elements 2a, 2b, . . . having strong directivity are used, the components irrelevant to the contribution to the ultrasonic beam US2 of the components included in the ultrasonic waves 2a′, 2b′ . . . , that is, unwanted components become small.

FIGS. 3A and 3B show acoustic pressure intensity profiles in the case where ultrasonic beams at large sound ray angles are transmitted. FIG. 3A shows the case of using plural elements having weak directivity, and FIG. 3B shows the case of using plural elements having strong directivity. As clearly seen from FIGS. 3A and 3B, in the case where ultrasonic beams are formed in the direction at large sound ray angles, the acoustic pressure intensity profile in which the acoustic pressure of the main lobe is larger can be obtained by using the element having weak directivity (FIG. 3A).

FIGS. 4A and 4B are diagrams for explanation of the principle of forming ultrasonic beams by using plural elements. An arc ARC3 and an arc ARC4 schematically show the drive timing with which plural elements 3a, 3b, . . . , and plural elements 4a, 4b, . . . are driven, respectively. As shown in FIGS. 4A and 4B, for example, in the case where ultrasonic beams US3 and US4 deflected toward the right direction of the drawings are formed, plural elements 3a, 3b, . . . , and plural elements 4a, 4b, . . . are driven sequentially from the left end toward the right end.

As shown in FIG. 4A, in the case where spherical waves 3a′, 3b′, . . . are generated from the elements 3a, 3b, . . . having weak directivity as described above, the components having nearly uniform intensity from all of the elements contribute to the formation of the ultrasonic beam US3 regardless of the arrangement of the elements 3a, 3b, . . .

On the other hand, as shown in FIG. 4B, ultrasonic waves 4a′, 4b′, . . . generated from the elements 4a, 4b, . . . having strong directivity includes only little amount of the components other than components in the direction perpendicular to the ultrasonic transducer array surface. On this account, when the sound ray angle of the ultrasonic beam is made larger, only little component becomes able to contribute to the formation of the ultrasonic beam US4. Accordingly, unwanted components that do not contribute to the formation of the ultrasonic beam US4 become relatively increased.

As described above, the relationship between the sound ray direction of the ultrasonic beam and the directivity of the plural ultrasonic wave components constituting the ultrasonic beam is closely associated with the formation of side lobes. Accordingly, in this embodiment, the directivity of the ultrasonic waves is controlled according to the sound ray direction of the ultrasonic beam. Because both are required to be adjusted in order to obtain the effect of reducing side lobes although the directivity can be determined arbitrarily to the sound ray direction.

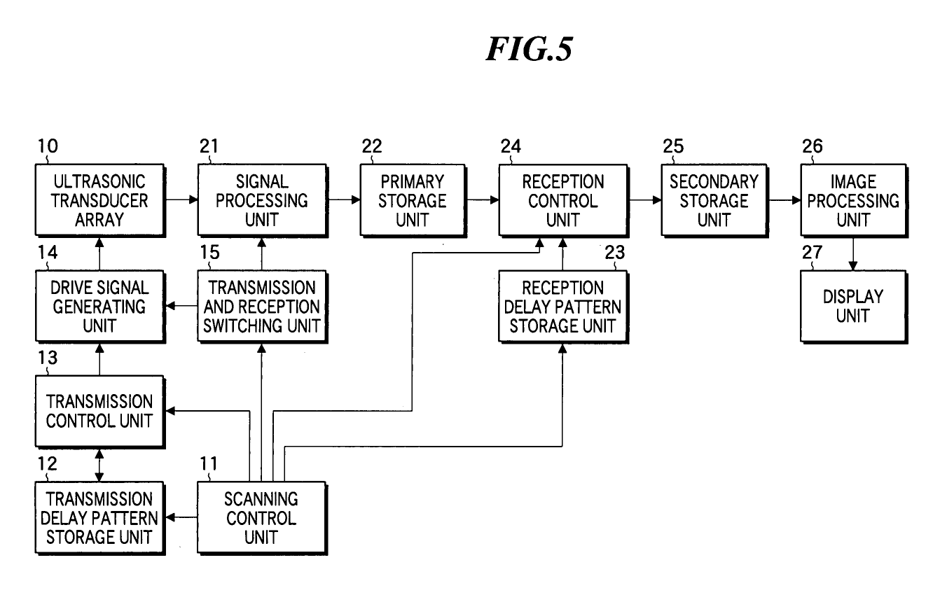

FIG. 5 is a block diagram showing a constitution of an ultrasonic transmitting and receiving apparatus according to the first embodiment of the present invention.

An ultrasonic transducer array 10 includes plural ultrasonic transducers (also referred to “elements”) arranged in a two-dimensional matrix form, for example, and, by controlling these ultrasonic transducers electronically, an object to be inspected is scanned electronically. The plural ultrasonic transducers transmit ultrasonic beams based on drive signals applied thereto, and receive ultrasonic waves reflected from the object to output detection signals. These ultrasonic transducers are constituted by a vibrator in which electrodes are formed on both ends of a material having a piezoelectric property (piezoelectric element) that includes piezoelectric ceramic represented by PZT (Pb (lead) zirconate titanate) or a macromolecule piezoelectric element represented by PVDF (polyvinylidene difluoride), for example. Applying a voltage to the electrodes of such vibrator by transmitting a pulsed electrical signal or continuous wave electrical signal, the piezoelectric element expands and contracts. By the expansion and contraction, pulsed or continuous ultrasonic waves are generated from the respective vibrators, and these ultrasonic waves are synthesized to form ultrasonic beams. Further, the respective vibrators expand and contract by receiving propagating ultrasonic waves and generate electrical signals. These electrical signals are outputted as detection signals of ultrasonic waves.

Alternatively, as the ultrasonic transducers, plural kinds of elements of different ultrasonic conversion methods may be used. For example, the above-described vibrator is used as an element for transmitting ultrasonic waves, and a photo-detection type ultrasonic transducer is used as an element for receiving ultrasonic waves. The photo-detection type ultrasonic transducer is for detecting an ultrasonic signal by converting it into an optical signal, and, for example, constituted by a Fabry-Perot resonator or fiber Bragg grating.

The ultrasonic transmitting and receiving apparatus according to this embodiment includes a scanning control unit 11, a transmission delay pattern storage unit 12, a transmission control unit 13, a drive signal generating unit 14, and a transmission and reception switching unit 15.

The scanning control unit 11 sequentially sets sound ray directions of transmitted and received ultrasonic beams. Further, the scanning control unit 11 controls respective units of the ultrasonic transmitting and receiving apparatus so that ultrasonic beams constituted by ultrasonic components having predetermined directivity may be transmitted and received in a preset direction.

The transmission delay pattern storage unit 12 has stored plural transmission delay patterns in which delay times to be provided to the plural elements are set, and plural patterns (directivity control patterns) for controlling directivity of the plural ultrasonic components constituting transmission beams. The transmission delay patterns and directivity control patterns are used when an ultrasonic beam is transmitted in the sound ray direction set by the scanning control unit 11. When transmitting an ultrasonic beam, a predetermined directivity control pattern is selected by the scanning control unit 11. By the way, the directivity control pattern will be described later in detail.

The transmission control unit 13 selects a predetermined transmission delay pattern from plural transmission delay patterns stored in the transmission delay pattern storage unit 12 based on the sound ray direction set by the scanning control unit 11. Further, the transmission control unit 13 sets the delay times to be provided to the plural elements included in the ultrasonic transducer array 10, respectively, based on the selected transmission delay pattern and directivity control pattern.

The drive signal generating unit 14 is constituted by, for example, plural pulsers corresponding to the plural elements, respectively. Each of the plural pulsers generates a drive signal based on the delay time set by the transmission control unit 13. Thereby, the transmission beam propagating toward the set direction is formed.

The transmission and reception switching unit 15 switches the generation of drive signals in the drive signal generating unit 14 and capture of the detection signals in a signal processing unit 21 with predetermined timing according to the control of the scanning control unit 11. By restricting the time periods for reading detection signals as described above, ultrasonic echo signals reflected from a particular depth of the object can be detected.

Further, the ultrasonic transmitting and receiving apparatus according to this embodiment includes the signal processing unit 21, a primary storage unit 22, a reception delay pattern storage unit 23, a reception control unit 24, a secondary storage unit 25, an image processing unit 26, and a display unit 27.

The signal processing unit 21 includes plural channels respectively corresponding to the plural elements. Each of the plural channels of the signal processing unit 21 captures the detection signal outputted from the corresponding element with predetermined timing, and performs signal processing such as logarithmic amplification, demodulation, STC (sensitivity time control), filtering processing, A/D conversion. The primary storage unit 22 includes plural lines respectively corresponding to the plural channels of the signal processing unit 21 and stores detection signals subjected to signal processing in the signal processing unit 21 with respect to each line in chronological order.

The reception delay pattern storage unit 23 has stored plural reception delay patterns in which delay times to be provided to the detection signals outputted from the plural elements are set, and the patterns for controlling the directivity of the plural ultrasonic components constituting reception beams. The reception delay patterns and directivity control patterns are used when reception focusing processing is performed so that the received ultrasonic reflected signals (echo signals) may form reception focal points in the predetermined sound ray direction and depth. When the ultrasonic echoes are received, a predetermined directivity control pattern is selected by the scanning control unit 11. By the way, the predetermined directivity control pattern will be described later in detail.

The reception control unit 24 selects a predetermined pattern from the plural reception delay patterns stored in the reception delay pattern storage unit 23 based on the sound ray direction set in the scanning control unit 11. Further, the reception control unit 24 performs reception focusing processing by providing delays to the plural detection signals respectively outputted from the plural elements based on the selected reception delay pattern and directivity control pattern and adding them. Thereby, sound ray data representing the reception beam with the focal point narrowed down in the set sound ray direction and depth is formed. The secondary storage unit 25 stores the sound ray data formed in the reception control unit 24.

The image processing unit 26 constructs two-dimensional or three-dimensional image data based on the sound ray data stored in the secondary storage unit 25, and performs image processing such as gain adjustment, contrast adjustment, gradation processing, response enhancement processing, interpolation processing on the image data. The display unit 27 displays ultrasonic images by scanning converting the image data image processed in the image processing unit 26. The display unit 27 includes a display device such as a CRT or an LCD, for example.

Next, directivity control patterns stored in the transmission delay pattern storage unit 12 and the reception delay pattern storage unit 23 will be described. Hereinafter, each of the plural ultrasonic components constituting an ultrasonic beam is referred to as “unit beam”.

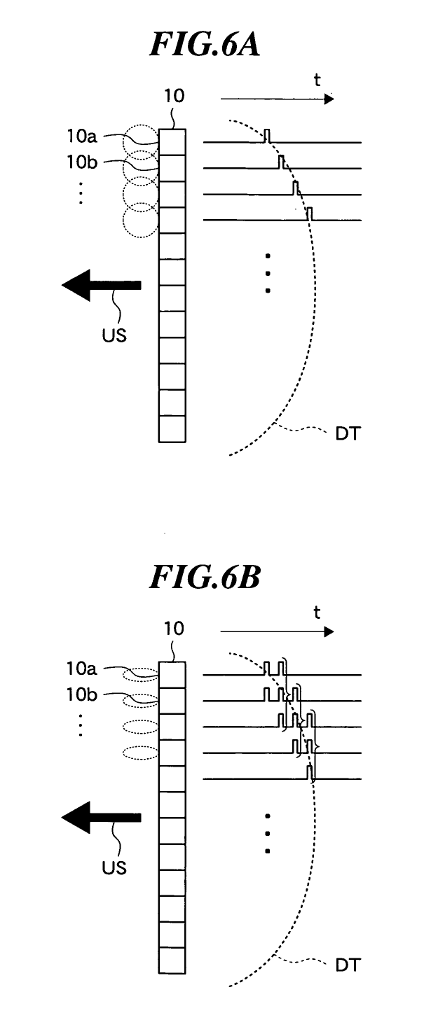

FIGS. 6A and 6B are diagrams for explanation of directivity control patterns. In FIGS. 6A and 6B, the section of the ultrasonic transducer array 10 and the timing for respectively driving the plural elements included therein are shown. Here, the delay pattern DT represents delay times to be provided to unit beams, respectively, when an ultrasonic beam is formed toward the direction shown by an arrow in the drawing.

In the case where the unit beam having weakest directivity is generated, the unit beam may be generated from one element. Accordingly, in this case, as shown in FIG. 6A, the plural elements 10a, 10b, . . . included in the ultrasonic transducer array 10 are sequentially driven based on the delay pattern DT. Thereby, spherical waves are generated from the elements 10a, 10b, . . . , respectively. Hereinafter, the method of generating unit beams by thus driving elements one by one is referred to as “normal drive method”. Note that the normal drive method is a method used in a general ultrasonic transducer array using an electrical sector scanning method.

In addition, in the case where the unit beam having strong directivity is generated, substantial aperture of the elements may be enlarged by driving the plural elements simultaneously. Accordingly, in this case, as shown in FIG. 6B, the simultaneously driven plural elements 10a, 10b, . . . are grouped in advance, and the respective groups are sequentially driven based on the delay patterns DT. In FIG. 6B, the elements are grouped so that three elements (10a, 10b, 10c), (10b, 10c, 10d), . . . may be included in each group. In order to strengthen directivity, the number of elements included in one group may be increased to enlarge the substantial aperture diameter of the elements that contribute to the formation of the unit beams. Simultaneously, by using the elements 10a, 10b, . . . in adjacent groups redundantly, the pitch of the unit beams can be made equal to that in the normal drive method. Hereinafter, the method for thus controlling directivity of the unit beams by changing the substantial aperture of the elements without changing the pitch of the unit beams is referred to as “effective aperture control method”.

The “directivity control pattern” means a pattern for thus grouping plural elements. This directivity control pattern is brought into correspondence with a sound ray direction so that the aperture of the unit beam may become broader when the sound ray angle is small, and the aperture of the unit beam may become narrower when the sound ray angle is large. The correspondence between the sound ray direction and the directivity control pattern may be defined so that the directivity control pattern may change in stages in response to the sound ray angle, or may change continuously.

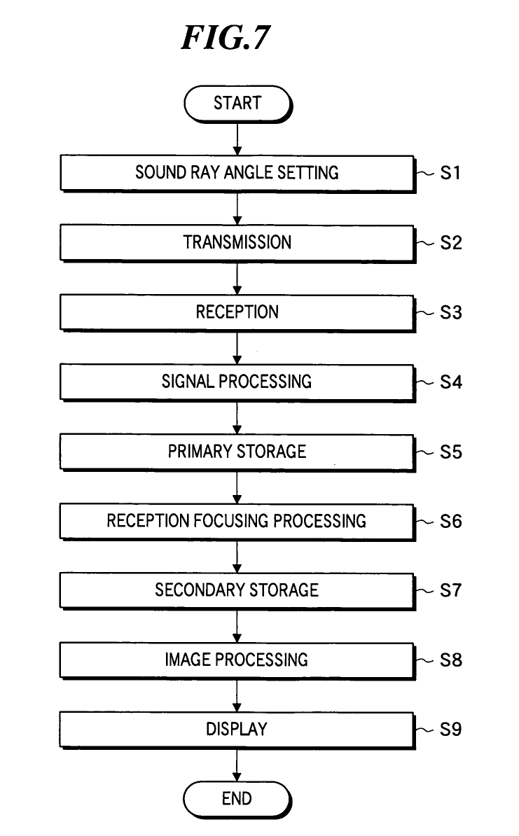

Next, the operation of the ultrasonic transmitting and receiving apparatus according to this embodiment will be described. FIG. 7 is a flowchart showing the operation of the ultrasonic transmitting and receiving apparatus according to this embodiment.

First, at step S1, the scanning control unit 11 sets sound ray angles of transmitted and received ultrasonic beams. Accordingly, the transmission control unit 13 sets delay times in the drive signal generating unit 14 based on the predetermined directivity control patterns and the transmission delay pattern.

At step S2, when the drive signal generating unit 14 generates drive signals. Thereby, plural unit beams having predetermined directivity are sequentially transmitted, and wavefront of those unit beams are synthesized to transmit the ultrasonic beam with respect to the sound ray direction set at step S1 is transmitted.

At step S3, the transmission and reception switching unit 15 is switched, and the ultrasonic transducer array 10 receives ultrasonic echoes. Each of the plural elements included in the ultrasonic transducer array 10 generates an electrical signal (detection signal) based on the received ultrasonic echo.

At step S4, the signal processing unit 21 performs signal processing such as logarithmic amplification, STC, filtering processing, A/D conversion on the detection signal outputted from each of the plural elements. At step S5, the signal processed detection signals (digital data) are sequentially stored in the primary storage unit 22.

At step S6, the reception control unit 24 performs reception focusing processing on the detection signals stored in the primary storage unit 22 based on the directivity control pattern and the transmission delay pattern corresponding to the sound ray direction set at step S1. Thereby, sound ray data representing the reception beam with respect to the sound ray direction set at step S1 is formed. The sound ray data formed at step S6 is stored in the secondary storage unit 25 (step S7).

At step S8, the image processing unit 26 constructs two-dimensional or three-dimensional image data based on the sound ray data stored in the secondary storage unit 25, and performs image processing such as gain adjustment and tone processing. Then, at step S9, the display unit 27 displays ultrasonic images on a display by scanning converting the image processed image data.

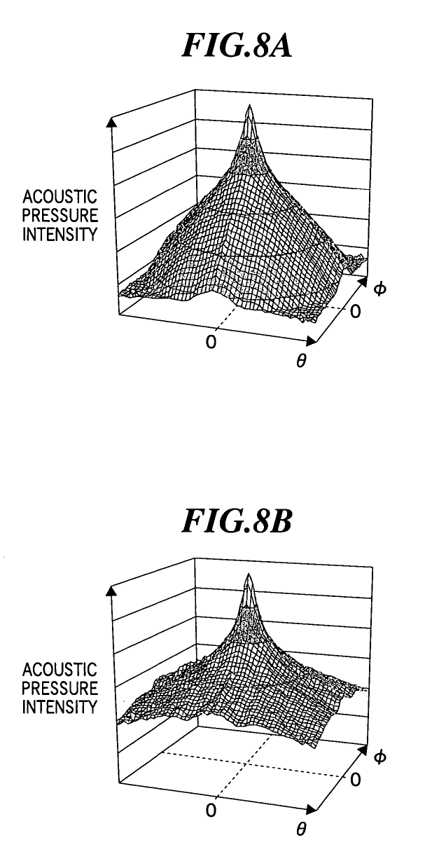

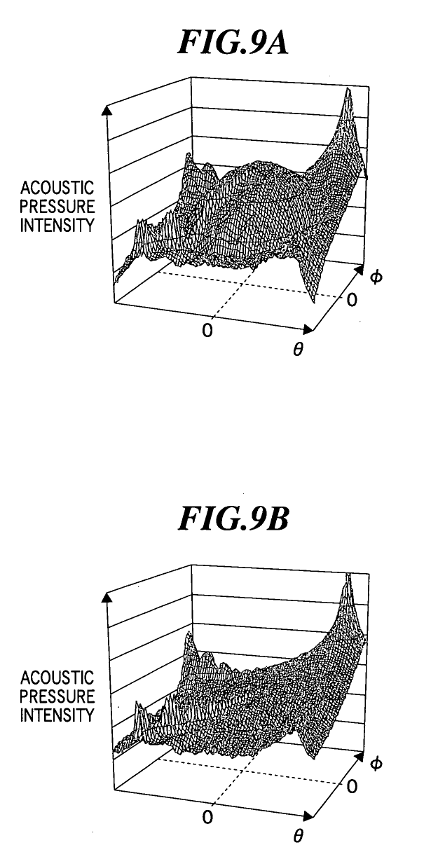

FIGS. 8A to 9B show acoustic pressure intensity profiles obtained by applying the ultrasonic transmitting and receiving apparatus according to this embodiment. These acoustic pressure intensity profiles are obtained by simulating acoustic pressure intensity distribution of transmission and reception under the condition that sine waves consisted of five wave train with center frequency of 2.5 MHz are transmitted from the ultrasonic transducer array in which plural elements of 0.35 mm square arranged at a 0.45 mm pitch.

FIG. 8A shows an acoustic pressure intensity profile in the case where the directivity of the unit beams is made strong by setting the effective aperture diameter of the elements to 1.4 mm so as to form an ultrasonic beam with sound ray angles θ=φ=0°. FIG. 8B shows an acoustic pressure intensity profile in the case where the directivity of the unit beams is made weak by setting the effective aperture diameter of the elements to 0.35 mm for comparison. As clearly seen from FIGS. 8A and 8B, when the sound ray angle is small, side lobe component is entirely reduced by strengthening the directivity of the unit beams.

FIG. 9A shows an acoustic pressure intensity profile in the case where the directivity of the unit beams is made strong by setting the effective aperture diameter of the elements to 1.4 mm so as to form an ultrasonic beam with sound ray angles θ=φ=32.5°. FIG. 9B shows an acoustic pressure intensity profile in the case where the directivity of the unit beams is made weak by setting the effective aperture diameter of the elements to 0.35 mm. As clearly seen by comparison between FIGS. 9A and 9B, when the sound ray angle is large, the intensity of the main lobe is maintained by weakening the directivity of the unit beams. Further, in FIG. 9B, there is no region where the side lobe component is large as in the vicinity of the center of FIG. 9A.

As described above, according to this embodiment, since the directivity of the unit beams is controlled in response to the sound ray direction, the side lobe component can be reduced over the entire of the scanning region. Further, according to this embodiment, in the effective aperture control method, since plural elements are used for forming one unit beam, there is an advantage that the sensitivity of the transmission beams and reception beams is improved.

In this embodiment, the directivity of the unit beams is controlled both when transmitting the ultrasonic beams and when performing reception focusing processing with respect to received ultrasonic echoes. However, by controlling the directivity of the unit beams in either transmission or reception, the side lobe component can be reduced.

In addition, when transmitting and receiving ultrasonic beams, different directivity control patterns may be used, respectively. At the time of transmission and reception, by changing the substantial aperture diameter of the elements for forming unit beams, different acoustic pressure intensity profiles of the reception beams are obtained with respect to the acoustic pressure intensity profiles of the transmission beams. Accordingly, for example, by selecting directivity control patterns so as to obtain acoustic pressure intensity profiles in which side lobe components cancel each other out in transmission and reception, the side lobe components can be entirely reduced.

Further, transmission and reception of ultrasonic waves may be performed by driving plural elements that contribute to one unit beam while weighting. Here, the weighting refers to weighting of intensity of waveforms for driving elements. Thereby, the directivity of unit beams can be made further stronger, and thus, the side lobe components can be further reduced. In the case of weighting, for example, weighting patterns have been stored in the transmission delay pattern storage unit 12 or the reception delay pattern storage unit 23, when the scanning control unit 11 selects a directivity control pattern, a weighting pattern is selected together and the multiplication thereof may be used. Alternatively, weighting may be performed on the entire of plural elements included in the ultrasonic transducer array 10. Further, the weighting on entire of the plural elements and the weighting on plural elements that form unit beams may be combined. As a weighting pattern, Gaussian distribution etc. are used.

Alternatively, the directivity control pattern may be created so that delay times may be generated between the grouped plural elements. Thereby, the directivity of unit beams can be made further stronger.

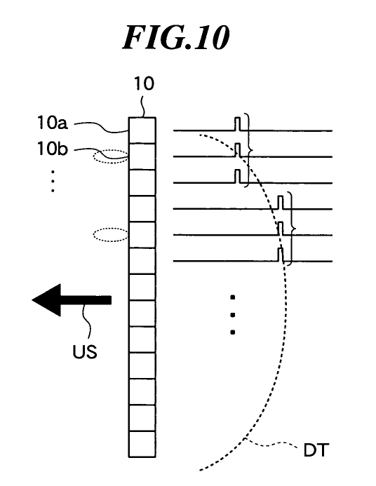

In this embodiment, when controlling the directivity of unit beams, the effective aperture control method as shown in FIG. 6B is used. However, in this method, there is a possibility that drive load on each element becomes larger because one element is driven in plural times within a short time. In such case, the aperture control method as shown in FIG. 10 may be used. The aperture control method is a method of controlling the directivity of unit beams by changing aperture diameter of elements, and, in the method, elements are never used redundantly between the unit beams formed adjacently. That is, as shown in FIG. 10, each element is included in only one group. Thereby, the drive load on the elements can be reduced, and the drive control system circuit can be simplified. According to the aperture control method, intervals between unit beams become larger compared to the case of the effective aperture control method. On this account, there is a possibility that grating lobes occur. Accordingly, it is desired that the intervals of unit beams, i.e., the number of elements to be grouped are set while considering the relationship with ultrasonic frequencies.

In this embodiment, the ultrasonic transducer array in which plural elements are arranged in a two-dimensional matrix form is used, however, the arrangement of elements is not limited to that, and an ultrasonic transducer array in which plural elements are arranged in other forms may be used. In addition, an ultrasonic transducer array in which plural elements are arranged one-dimensionally may be used.

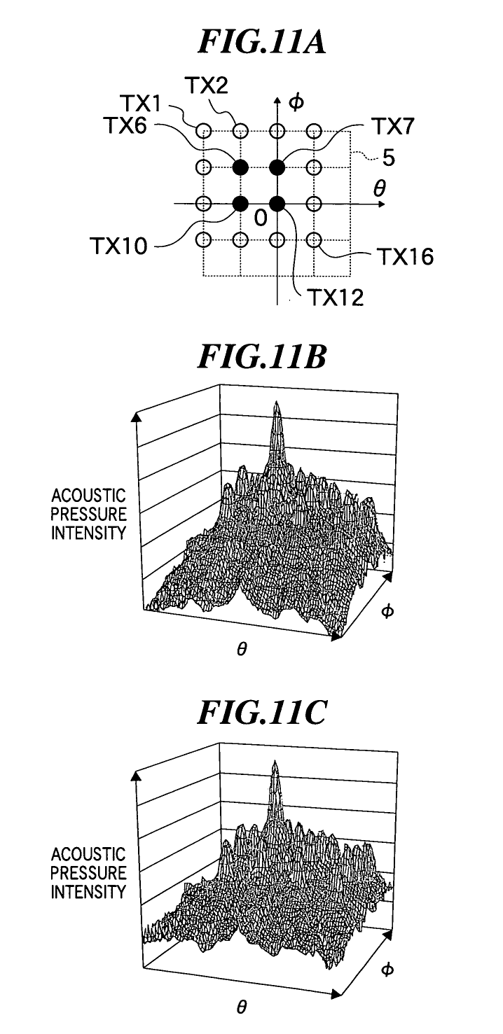

Next, an ultrasonic transmitting and receiving apparatus according to the second embodiment of the present invention will be described by referring to FIGS. 11A to 11C. This embodiment is to control the directivity of unit beams in the multi-beam transmission and reception for forming plural ultrasonic beams simultaneously in different plural directions. The constitution of the apparatus is the same as that shown in FIG. 5.

FIG. 11A shows plural sound ray directions formed with the array center of the ultrasonic transducer array as θ=φ=0°. As shown in FIG. 11A, the case where 16 ultrasonic beams TX1 to TX16 are transmitted simultaneously toward a scanning region 5, and reception focusing processing is performed with respect to one direction will be discussed. In this case, four ultrasonic beams TX6, TX7, TX10, and TX11 transmitted to the vicinity of the center where sound ray angles are small are formed by unit beams having strong directivity, and the ultrasonic beams transmitted in other directions are formed by unit beams having weak directivity.

FIG. 11B shows an acoustic pressure intensity profile obtained by performing simulation under such settings. Further, FIG. 11C shows an acoustic pressure intensity profile in the case where all of the ultrasonic beams are formed by unit beams having weak directivity for comparison. As clearly seen from FIGS. 11B and 11C, the side lobe components are further reduced entirely in the case where unit beams having different directivity are combined in response to the sound ray directions. Especially, in the direction at large sound ray angles, the difference is remarkable.

Generally, in the case of performing multi-beam transmission and reception, there is a tendency that side lobe components become higher as compared to the case where ultrasonic beams are formed in single sound ray direction. However, according to this embodiment, since the directivity of unit beams that form respective ultrasonic beams is controlled in response to the sound ray direction, the entire imaging region can be scanned at high speed while reducing side lobe components over broader range.

In this embodiment, the case where plural ultrasonic beams are transmitted in plural directions, respectively, and reception focusing is performed so as to form one reception focal point with respect to received ultrasonic echoes has been described. However, reception focusing processing may be performed so as to form reception focal points in plural directions with respect to received ultrasonic echoes. For example, reception focusing processing may be performed so as to form plural reception focal points in directions corresponding to plural transmission beams, respectively. In this case, it is desired also when reception focusing processing is performed that the directivity of unit beams is controlled in response to the sound ray directions in which reception focal points are formed.

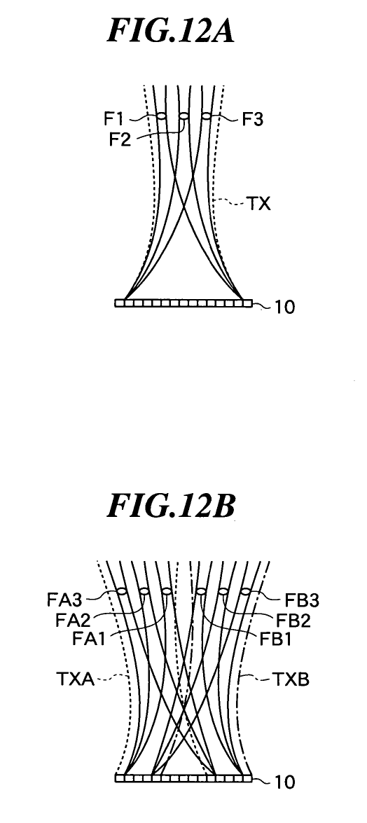

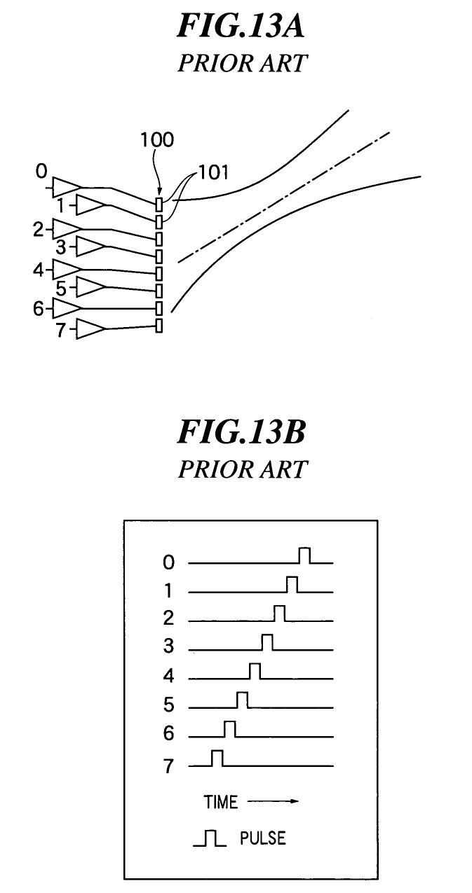

Further, as shown in FIG. 12A, reception focusing processing may be performed by transmitting an ultrasonic beam TX in single direction so as to form reception focal points F1 to F3 in plural directions. At that time, it is desired that the directivity of unit beams that form the transmission beam is controlled in response to the sound ray directions in which reception focal points are formed. Further, when performing reception focusing processing, directivity of unit beams may be controlled. In this case, since the ultrasonic beam is transmitted in single direction, the side lobe component can be suppressed compared to the case of multi-beam transmission. Moreover, since plural reception focal points are formed with respect to the received ultrasonic echoes, the imaging region can be scanned at high speed.

Furthermore, as shown in FIG. 12B, plural ultrasonic beams TXA and TXB may be transmitted in plural directions, respectively, and reception focusing processing may be performed on these transmission beams TXA and TXB so as to form reception focal points FA1 to FA3 and FB1 to FB3 in plural directions. In this case, in order to suppress side lobe components, it is desired that intervals of the plural ultrasonic beams TXA and TXB are separated from each other. At that time, the directivity of unit beams that form the transmission beam is controlled in response to the sound ray directions, respectively. The directivity of unit beams may be controlled also when performing reception focusing processing.

As described above, according to the present invention, the directivity of plural unit beams that form ultrasonic beams is controlled in response to sound ray directions. Thereby, side lobe components are reduced over the entire scanning region, and detection signals with high S/N ratios can be obtained. Therefore, based on such detection signals, ultrasonic images with high image quality can be acquired.

1. An ultrasonic transmitting and receiving apparatus comprising:
an ultrasonic transducer array including plural ultrasonic transducers for transmitting ultrasonic waves and receiving ultrasonic waves reflected from an object to be inspected;
drive signal generating means for generating drive signals for respectively driving said plural ultrasonic transducers;
transmission control means for controlling said drive signal generating means such that ultrasonic waves to be transmitted from said plural ultrasonic transducers form a transmission beam to be transmitted in at least one direction;
reception control means for performing reception focusing processing on plural detection signals obtained based on ultrasonic waves received by said plural ultrasonic transducers so as to form a reception focal point in at least one direction thereby forming a reception beam; and
control means for changing directivity of plural ultrasonic components constituting the transmission beam in accordance with a sound ray direction in which the transmission beam is transmitted and/or changing directivity of plural ultrasonic components constituting the reception beam in accordance with a sound ray direction in which the reception focal point of the receiving beam is formed.

2. An ultrasonic transmitting and receiving apparatus according to claim 1, wherein said control means makes the directivity of said ultrasonic components stronger as an angle formed by a front direction of said ultrasonic transducer array and said sound ray direction becomes smaller.

3. An ultrasonic transmitting and receiving apparatus according to claim 1, wherein said control means changes the directivity of said ultrasonic components by changing a number of ultrasonic transducers to be used simultaneously when forming said ultrasonic components.

4. An ultrasonic transmitting and receiving apparatus according to claim 2, wherein said control means changes the directivity of said ultrasonic components by changing a number of ultrasonic transducers to be used simultaneously when forming said ultrasonic components.

5. An ultrasonic transmitting and receiving apparatus according to claim 1, wherein said control means performs weighting on the plural drive signals to be used when forming said ultrasonic components.

6. An ultrasonic transmitting and receiving apparatus according to claim 2, wherein said control means performs weighting on the plural drive signals to be used when forming said ultrasonic components.

7. An ultrasonic transmitting and receiving apparatus according to claim 3, wherein said control means performs weighting on the plural drive signals to be used when forming said ultrasonic components.

8. An ultrasonic transmitting and receiving apparatus according to claim 4, wherein said control means performs weighting on the plural drive signals to be used when forming said ultrasonic components.

9. An ultrasonic transmitting and receiving apparatus according to claim 1, wherein said reception control means performs reception focusing processing on said at least one direction in which the transmission beam is transmitted so as to form reception focal points in plural directions.

10. An ultrasonic transmitting and receiving apparatus according to claim 2, wherein said reception control means performs reception focusing processing on said at least one direction in which the transmission beam is transmitted so as to form reception focal points in plural directions.

11. An ultrasonic transmitting and receiving apparatus according to claim 3, wherein said reception control means performs reception focusing processing on said at least one direction in which the transmission beam is transmitted so as to form reception focal points in plural directions.

12. An ultrasonic transmitting and receiving apparatus according to claim 4, wherein said reception control means performs reception focusing processing on said at least one direction in which the transmission beam is transmitted so as to form reception focal points in plural directions.

13. An ultrasonic transmitting and receiving apparatus according to claim 5, wherein said reception control means performs reception focusing processing on said at least one direction in which the transmission beam is transmitted so as to form reception focal points in plural directions.

14. An ultrasonic transmitting and receiving apparatus according to claim 1, wherein said reception control means performs reception focusing processing on plural directions in which the transmission beams are transmitted so as to form reception focal points in the plural directions, respectively.

15. An ultrasonic transmitting and receiving apparatus according to claim 2, wherein said reception control means performs reception focusing processing on plural directions in which the transmission beams are transmitted so as to form reception focal points in the plural directions, respectively.

16. An ultrasonic transmitting and receiving apparatus according to claim 3, wherein said reception control means performs reception focusing processing on plural directions in which the transmission beams are transmitted so as to form reception focal points in the plural directions, respectively.

17. An ultrasonic transmitting and receiving apparatus according to claim 4, wherein said reception control means performs reception focusing processing on plural directions in which the transmission beams are transmitted so as to form reception focal points in the plural directions, respectively.

18. An ultrasonic transmitting and receiving apparatus according to claim 5, wherein said reception control means performs reception focusing processing on plural directions in which the transmission beams are transmitted so as to form reception focal points in the plural directions, respectively.
